(19) United States
(12) Patent Application Publication
(10) Pub. No.: US 2003/0199728 A1
(43) Pub. Date: Oct. 23, 2003

(54) Agent for enhancing alpha-wave appearance, an agent for inducing a relaxed state and a food or drink comprising the agent

(76) Inventors: Jun Kashimura, Kanagawa (JP); Yoshio Machi, Saitama (JP)

Correspondence Address: Gerald Levy, Esq., PITNEY, HARDIN, KIPP & SZUCH LLP, 685 Third Avenue, New York, NY, 10017-4024, US

(21) Appl. No.: 10/418,606

(22) Filed: Apr. 18, 2003

(30) Foreign Application Priority Data
Apr. 19, 2002 (JP) 2002-117907
Apr. 15, 2003 (JP) 2003-110635

(51) Int. Cl. A61M 21/00

(52) U.S. Cl. 600/26

(57) ABSTRACT

The invention provides an agent comprising palatinose, for enhancing appearance of α wave, particularly, α 1 wave, and inducing a relaxed state to thereby promote alleviation or relief of mental stress or tense.

### FIELD OF THE INVENTION

This invention relates to an agent able to induce a relaxed state by enhancing appearance of α wave, which is one of brain waves of a human being and relates also to a food or drink comprising the agent.

### PRIOR ART

Brain waves of human being are being classified by frequency, broadly into four categories: δ wave whose frequency is less than 4 Hz, θ wave whose frequency is no less than 4 Hz and less than 8 Hz, α wave whose frequency is no less than 8 Hz and less than 13 Hz, and β wave whose frequency is no less than 13 Hz. It is known that among these brain waves, α wave is found more in a state of relaxation, while it is less in a stressed state. Accordingly, α wave generation is considered to be an effective index for a relaxed state. Further, α wave is classified into α 1 wave whose frequency is no less than 8 Hz and less than 10 Hz and α 2 wave whose frequency is no less than 10 Hz and less than 13 Hz, and both are considered an index of a relaxed state.

In the contemporary society, people are often imposed on mental stress or tense. Consequently, physical and mental relaxation is intensively required in order to sustain people's physical and mental health.

As a conventional means to attain physical and mental relaxation, there are known ways to enhance appearance of α wave via the sense of hearing or sight, e.g., a bio-feedback method, α wave music, and light feedback devices. However, these ways have drawbacks that in order to induce a relaxed state by enhancing appearance of α wave through these ways, a special device or a special place which is suitable for the use of the device is needed and, further, special training is necessary to use the device.

As a means to realize physical and mental relaxation by ingesting food, it is also known that appearance of α wave is enhanced by ingesting herb tea such as chamomile tea or hyssop tea leading to a relaxed state (Shokuhinkogyo. Korin, Co. Ltd., 44 (10), 23-27, Tsunoda, Hideo, May 30, 2001). It is also known that α wave appearance is enhanced by ingesting theanine, a component of green tea (JP Publication 09-12454/1997, pages 1-5) or maracuja juice (JP Publication 07-126179/1995, pages 1-6), leading to a relaxed state. However, these means have problems that application is limited due to likes and dislikes caused by unique flavor or taste of herb tea, or the manufacturing process of theanine or maracuja juice is complicated or costly. If it is possible to easily enhance appearance of α wave to induce a relaxed state without any special devices, this is very useful in sustaining physical and mental health. In this background, it is desirable to solve the aforesaid problems by using a material which is edible and available easily at low cost.

### SUMMARY OF THE INVENTION

The present inventors have found that it is possible to enhance appearance of α wave to thereby induce a relaxed state by ingesting palatinose, which is food, and have attained the invention.

The invention provides an agent for enhancing appearance of α wave comprising palatinose. The agent for enhancing α wave appearance can enhance appearance of α wave to induce a relaxed state or to generate a state of relaxation.

The invention provides an agent for enhancing appearance of α 1 wave, one of α waves, comprising palatinose. The agent for enhancing the α 1 wave appearance can enhance appearance of α 1 wave to induce a relaxed state or to generate a state of relaxation.

The invention also provides an agent for inducing a relaxed state, which comprises palatinose.

Further, the invention provides a food or drink comprising the agent for enhancing appearance of α wave, the agent for enhancing appearance of α 1 wave, or the agent able to induce a relaxed state.

### PREFERRED EMBODIMENTS OF THE INVENTION

In the description of the invention, “α wave” refers to a brain wave of human being and animals in a frequency of no less than 8 Hz and less than 13 Hz, and “α 1 wave”, a kind of α wave, refers to a brain wave of human being or animal in a frequency of no less than 8 Hz and less than 10 Hz.

Alpha wave can be measured by referential derivation method. Illustrative examples will be mentioned below for methods of measurement, record, and analysis, but the invention is not restricted by them and any known methods can be employed.

Brain waves can be measured in accordance with the international 10-20 standard electrode positioning system recommended by the International Electroencephalography Association, by locating electrodes on 19 positions on the scalp and further locating a reference electrode on each earlobe. For measuring and recording electroencephalogram, Electroencephalograph 5500 manufactured by NEC San-ei Co., Ltd. can be used.

For analyzing electroencephalogram, topographic mapping is performed using a potential mapping process on α wave. Then a ratio of an area occupied by an α wave appearance part to the entire area of topogram is calculated and the ratios are averaged over all of the topograms to obtain an average α wave appearance ratio. For the electroencephalogram analysis, ATALAS, manufactured by Kissei Comtec Co., Ltd., can be employed.

Enhancement of α wave appearance can be judged by the following tests which are not limitative.

For each subject, an average α wave appearance ratio is obtained in a resting state with his or her eyes closed before the subject takes a drink comprising the agent according to the invention; and an average α wave appearance ratio in a resting state with his or her eyes closed after the subject takes and does work. Then, appearance of α wave can be evaluated by a ratio of the latter ratio to the former ratio, i.e., an average α wave appearance-enhancing ratio.

The reason why α wave is measured after work is that α wave decreases when a person is imposed with a workload, and then it is easier to observe a change in α wave. An example of the work is a word processing work, for example, inputting sentences described in a book into a word processor. In the inputting, it is recommendable to select pages to be input at random, for each subject and each run. The reason is that if a same subject inputs same sentences repeatedly, he/she might memorize the sentences, which may affect the results of electroencephalogram.

When electroencephalogram measurement is conducted on a material having sweet taste, it is recommendable to equalize a degree of sweetness so as not to give subjects a prejudice resulting from difference in sweetness. In experiments by the inventors, it has been found that a mixture of aspartame and acesulfame potassium, both high intensity sweetener, does not affect an α wave enhancing effect (see the preliminary test below). Accordingly, it is possible to employ a mixture of aspartame and acesulfame potassium in order to equalize a degree of sweetness.

In the description of the invention, the phrase, “to be able to induce a relaxed state” or “to be able to generate a relaxed state”, means that it is possible to enhance α wave, preferably α 1 wave.

The agent for enhancing α wave appearance or the agent able to induce a relaxed state in accordance with the invention comprises palatinose as an active ingredient.

Palatinose, which is also called isomaltulose, is a disaccharide in which glucose is bonded to fructose via α-1,6-glucosyl bond. The properties of palatinose monohydrate crystal are as follows: melting point, 123-124° C.; specific rotation, [α]_{D}²⁰+97.2; reducing power to Fehling's solution, 52% of glucose; solubility in 100 g of water, 38.4 g at 20° C., 78.2 g at 40° C., and 174.9 g at 60° C.; quality of sweet taste of its aqueous solution, good; and the degree of sweetness, about 40% of sucrose.

Palatinose is found in honey and cane juice in nature. It is also present in a rearrangement product which is formed when α-glucosyltransferase, isomaltulose synthase, originating from a bacterium or yeast, acts on sucrose.

Commercially, sucrose is converted mostly into palatinose by subjecting sucrose to the action of α-glucosyltransferase which a bacterium, such as Protaminobacter rubrum or Serratia plymuthica, produces.

The agent for enhancing α wave appearance or the agent able to induce a relaxed state according to the invention may have a form such as crystalline palatinose, and syrup such as palatinose syrup or trehalulose syrup. Crystalline palatinose (Crystalline Palatinose-IC, trade name, Shin Mitsui Sugar Co., Ltd.) contains 99.0% or more of palatinose including crystal water. Palatinose syrup (Palatinose Syrup-ISN or -TN, trade name, Shin Mitsui Sugar Co., Ltd.) contains 11-17% of palatinose and some trehalulose. Trehalulose syrup (Mildear-75 or -85, trade name, Shin Mitsui Sugar Co., Ltd.) contains 8-13% of palatinose and some trehalulose.

The agent for enhancing α wave appearance or the agent able to induce a relaxed state in accordance with the present invention also includes, for instance, fondants, granules, tablets, and syrup comprising crystalline palatinose or palatinose syrup, and powder mixtures comprising crystalline palatinose or palatinose syrup with any acidulants, sweeteners, sugar esters, or flavors.

The food or drink comprising the agent for enhancing α wave appearance or the agent able to induce a relaxed state according to the present invention includes various types of beverage, e.g., soft drinks, near water, sports drinks, jelly drinks, and coffee drinks, and various types of food, e.g., soft jelly, hard candy, and chocolate. The agent for enhancing α wave appearance or the agent able to induce a relaxed state according to the invention may be added as a sweetener to coffee, tea, or cocoa, alone or together with other saccharides or high intensity sweeteners.

It is preferred that the agent for enhancing α wave appearance or the agent able to induce a relaxed state according to the invention is taken in an amount of five grams or more, more preferably 10 grams or more of palatinose at one time or in short intervals. For example, in the case of canned juice of 250 mL comprising 12 g of palatinose, a can of juice may be taken at one time. In the case of candy containing 4-5 g of palatinose, 2 or 3 candies may be eaten at one time. In the case of a jelly drink containing 15 g of palatinose, one jelly drink may be taken at one time. These are enough to attain relaxation effect in accordance with the present invention.

Since ingestion of the agent for enhancing α wave appearance, the agent able to induce a relaxed state, or the food or drink comprising the agent can enhance appearance of α wave and induce a relaxed state, it is possible to promote relief or alleviation of mental stress or tense.

The invention will be explained in more detail with reference to the Examples, but the invention shall not be limited by these Examples.

### EXAMPLES

In the preliminary test and Example 1 described below, Electroencephalograph 5500 manufactured by NEC San-ei Co., Ltd was used for measurement and record of electroencephalogram and ATALAS manufactured by Kissei Comtec. Co., Ltd. was employed for electroencephalogram analysis.

As mentioned above, the degree of sweetness of palatinose is 40, given that the degree of sweetness of sucrose as control is 100. Accordingly, in Example 1 described below, the intake weight of palatinose was set equal to that of sucrose as a control sample, and a mixture of aspartame and acesulfame potassium, high intensity sweetener, was added to a palatinose sample (or drink) to attain the same degree of a sweetness as that of sucrose sample (or drink) (hereinafter referred to as “control sample”).

That a mixture of aspartame and acesulfame potassium does not affect an α wave appearance enhancing effect will be demonstrated below first.

A drink was prepared by dissolving 0.1 g of aspartame and 0.1 g of acesulfame potassium in distilled water to attain a total weight of 190 g. The added amounts of aspartame and acesulfame potassium are to give the same degree of sweetness as that of a drink where 40 g of sucrose is dissolved in 150 g of distilled water.

Three healthy males aged 21 to 23 (subjects J, K, and L described in Example 1 below) were chosen as subjects. Whether electroencephalogram of each subject be normal or not was checked by measuring the entire electroencephalogram prior to the test as well as during the test by simultaneous monitoring.

Each subject did not take breakfast on the test day to be in a fasting state for 12 hours or more before the test. According to the international 10-20 standard electrode positioning system recommended by the International Electroencephalography Association, each subject was equipped with 19 electrodes on the scalp and further with each reference electrode on each earlobe. First, electroencephalogram of each subject was taken for five minutes in a resting state with his eyes closed. Then each subject ingested 190 g of the above-mentioned drink at one time. Each subject performed a word processing work for 20 minutes 130 minutes after ingesting the drink. Then electroencephalogram was taken for three minutes in a resting state with his eyes closed according to the above-described method.

In the word processing work, the subject input sentences of a book written in Japanese. The pages to be input were selected at random.

For electroencephalogram analysis, topographic mapping was performed on α 1 wave using a potential mapping process. From the topogram, an average α 1 wave appearance ratio in a resting state with closed eyes before the subject took the drink and that after the subject took the drink and did the work were obtained to calculate an average α 1 wave appearance enhancing ratio of the subject. As a result, the average α 1 wave appearance enhancing ratios for the three subjects were 1.06, 1.08, and 1.06, respectively. From the result, it was confirmed that a mixture of aspartame and acesulfame potassium did not affect the α wave appearance enhancing effect.

### Example 1

A drink comprising the agent according to the invention was prepared by dissolving 40 g of the present agent crystalline palatinose (Crystalline Palatinose-IC, trade name Shin Mitsui Sugar Co., Ltd.), 0.05 g of aspartame, and 0.05 g of acesulfame potassium were dissolved in distilled water with a total weight of the solution of 190 g (hereinafter referred to as “the drink of Example 1”).

The added amounts of aspartame and acesulfame potassium are such that a degree of sweetness of the drink of Example 1 is same as that of the control sample.

Eleven healthy males and one healthy female aged 21 to 40 were selected as subjects. Whether electroencephalogram of each subject be normal or not was checked by measuring the entire electroencephalogram prior to the test as well as during the test by simultaneous monitoring.

Each subject received electroencephalogram measurement as mentioned above.

On another day, each subject received electroencephalogram measurement as described above, but with 190 g of control sample.

The results are as seen in Table 1.

TABLE 1
Average α 1 Wave Appearance Enhancing Ratio

Average α 1 Wave Appearance
Enhancing Ratio After Work

| | Control Sample | Test Sample |
|---|---|---|
| Subject A | 1.12 | 1.13 |
| Subject B | 0.61 | 0.81 |
| Subject C | 1.10 | 1.15 |
| Subject D | 0.93 | 1.01 |
| Subject E | 1.32 | 1.39 |
| Subject F | 1.03 | 1.05 |
| Subject G | 1.02 | 1.02 |
| Subject H | 1.02 | 1.02 |
| Subject I | 1.42 | 1.72 |
| Subject J | 1.02 | 1.24 |
| Subject K | 1.22 | 1.43 |
| Subject L | 1.33 | 1.54 |

Test Results

As seen in Table 1, appearance of α 1 wave increased more when the test sample (drink of Example 1) was ingested than when the control sample (aqueous solution of sucrose) was ingested. Using t-test for paired samples, significant difference test was conducted on average α 1 wave appearance enhancing ratio between the control sample group (subjects A-L) and the test sample group (subjects A-L). As a result, the difference is recognized as significant with 1% or less of a risk rate. Thus, appearance of α 1 wave is effectively enhanced and a relaxed state can be attained by ingesting the drink comprising palatinose.

### Example 2

An agent according to the present invention, tablets, was manufactured according to the following recipe. Mixed powder of the following recipe was compressed at a tablet pressure of 300 kg per cm² to form tablets each of which had a diameter of 18 mm, thickness of 5 mm, and weight of 1.5 g.

| | | |
|---|---|---|
| Palatinose powder | 55 | parts by weight |
| (Palatinose Powder - ICP, trade name, | | |
| Shin Mitsui Sugar Co., Ltd) | | |
| Citric acid | 1 | part by weight |
| Sugar ester | 1 | part by weight |
| Aspartame | 0.05 | parts by weight |
| Vitamin P | 0.0002 | parts by weight |
| Water | 0.6 | parts by weight |
| Lemon flavor | proper | amount |

### Example 3

An agent according to the present invention, fondant, was manufactured in the following recipe. Crystalline palatinose was fed through a raw material feeder of a twin-screw extruder at a rate of 120 kg per hour and melted at 160-200° C. Subsequently, water was fed for cooling at a rate of 5.6 kg per hour, and then microcrystals were formed. Finally, palatinose syrup was fed at a rate of 100 kg per hour and mixed under cooling.

| | | |
|---|---|---|
| Crystalline palatinose | 120 | parts by weight |
| (Crystalline Palatinose - IC, trade name, | | |
| Shin Mitsui Sugar Co., Ltd.) | | |
| Palatinose syrup (Bx. 75) | 100 | parts by weight |
| (Palatinose Syrup - ISN, trade name, | | |
| Shin Mitsui Sugar Co., Ltd.) | | |

### Example 4

A sport drink containing the agent according to the invention was manufactured in the follow recipe. The following ingredients were dissolved in 215 mL of hot water and filled in a drink can for 250 mL.

| | | |
|---|---|---|
| Palatinose syrup (Bx. 75) | 50.0 | g |
| (Palatinose Syrup - ISN, trade name, | | |
| Shin Mitsui Sugar Co., Ltd.) | | |
| Vitamin C | 0.075 | g |
| Vitamin B₁ hydrochloride | 0.005 | g |
| Sodium citrate | 0.255 | g |
| Magnesium dichloride | 0.03 | g |
| Calcium lactate | 0.03 | g |
| Citric acid anhydride | 0.36 | g |
| Flavor | 0.03 | g |

### Example 5

Hard candies containing the agent according to the present invention were manufactured in the following recipe. Crystalline palatinose, palatinose syrup, and water were put in a dissolver and heated to dissolve under stirring. They were heated at a temperature of 120° C. at a reduced pressure of 86.7 kPa Gauge (vacuum degree: 650 mmHg), and then aspartame, citric acid, tartaric acid, red color, blue color, and grape flavor were added and mixed. After cooled to about 70-80° C., the mixture was shaped into candies so that each piece would weigh 4 g, which was packaged separately.

| | | |
|---|---|---|
| Crystalline palatinose | 50 | parts by weight |
| (Crystalline Palatinose - IC, trade name, | | |
| Shin Mitsui Sugar Co., Ltd.) | | |
| Palatinose syrup (Bx. 75) | 50 | parts by weight |
| (Palatinose Syrup - ISN, trade name, | | |
| Shin Mitsui Sugar Co., Ltd.) | | |
| Grape flavor | 0.25 | parts by weight |
| (No. 6 - 6240, trade name, | | |
| T. Hasegawa Co., Ltd.) | | |
| Red color | 0.10 | parts by weight |
| (TH - L, trade name, | | |
| T. Hasegawa Co., Ltd.) | | |
| Blue color | 0.05 | parts by weight |
| (TEl - 3L, trade name, | | |
| T. Hasegawa Co., Ltd.) | | |
| Citric acid | 1.00 | parts by weight |
| Tartaric acid | 0.30 | parts by weight |
| Aspartame | 0.12 | parts by weight |
| Water | 10 | parts by weight |

### Example 6

Hard candies containing the agent according to the present invention were manufactured in the following recipe. Crystalline palatinose and water were put in a dissolver and heated to dissolve under stirring. They were heated at a temperature of 120° C. at a reduced pressure of 86.7 kPa Gauge (vacuum degree: 650 mmHg), and then aspartame, citric acid, yellow color and lemon flavor were mixed. After cooled to about 70-80° C., the mixture was shaped into candies so that each piece would weigh 4 g, which was packaged separately.

| | | |
|---|---|---|
| Crystalline palatinose | 80 | parts by weight |
| (Crystalline Palatinose - IC, trade name, | | |
| Shin Mitsui Sugar Co. Ltd.) | | |
| Citric acid | 1.56 | parts by weight |
| Aspartame | 0.05 | parts by weight |
| Lemon flavor | 0.15 | parts by weight |
| (No. 6 - 6389, trade name, | | |
| T. Hasegawa Co., Ltd) | | |
| Yellow color | 0.04 | parts by weight |
| (TH - S, trade name, | | |
| T. Hasegawa Co., Ltd.) | | |
| Water | 18.2 | parts by weight |

### Example 7

A jelly drink with orange taste containing the agent according to the present invention was manufactured in the following recipe. First, palatinose syrup and water were mixed with each other, to which a selling agent was added portionwise to dissolve under heating up to 90° C. Then, the mixture was cooled to 70° C., to which the remaining ingredients were added and mixed to dissolve. The solution was filled in a cheer pack, sealed, and sterilized at a temperature of 90° C. for 20 minutes, and then cooled.

| | | |
|---|---|---|
| Palatinose (Bx. 75) | 15 | parts by weight |
| (Palatinose Syrup - ISN, trade name, | | |
| Shin Mitsui Sugar Co., Ltd.) | | |
| Gelling agent | 1 | part by weight |
| Orange fruit juice, 1/5 concentrated | 4 | parts by weight |
| Water | 80 | parts by weight |
| Citric acid | 0.35 | parts by weight |
| Sodium citrate | 0.2 | parts by weight |
| Vitamin C | 0.6 | parts by weight |
| β - Carotene | 0.01 | parts by weight |
| Stevia Sweetner | 0.01 | parts by weight |
| Vitamin P | 0.0004 | parts by weight |
| Orange flavor | proper | amount |

1. An agent for enhancing α wave appearance, comprising palatinose.

2. An agent for enhancing α wave appearance according to claim 1, wherein α wave is α 1 wave.

3. An agent for inducing a relaxed state, comprising palatinose.

4. A food or drink comprising the agent according to any one of claims 1-3.

5. Use of palatinose for manufacturing an agent for enhancing α wave appearance.

6. Use according to claim 5, wherein α wave is α 1 wave.

7. Use according to claim 5, wherein an agent for enhancing α wave appearance is a food or drink.

8. Use of palatinose for manufacturing an agent able to induce a relaxed state.

9. Use according to claim 8, wherein an agent able to induce a relaxed state is a food or drink.
